(19) United States
(12) Patent Application Publication
(10) Pub. No.: US 2009/0018621 A1
(43) Pub. Date: Jan. 15, 2009

(54) Medical and/or Cosmetic Radiation Device

(75) Inventors: Klaus Vogler, Eckental (DE); Daniel Haberer, Forchheim (DE); Heiko Kallert, Emskirchen (DE)

Correspondence Address: HAYNES AND BOONE, LLP;IP Section, 2323 Victory Avenue, Suite 700, Dallas, TX, 75219, US

(73) Assignee: Wavelight Laser Technologie AG, Erlangen (DE)

(21) Appl. No.: 11/816,400

(22) PCT Filed: Feb. 7, 2006
(86) PCT No.: PCT/EP06/01081

§ 371 Date: Apr. 2, 2008

(30) Foreign Application Priority Data
Feb. 16, 2005 (EP) 05003324.0

(51) Int. Cl. F21V 9/00; A61N 5/06

(52) U.S. Cl. 607/90; 362/231

(57) ABSTRACT

A medical and/or cosmetic radiation device has a plurality of LEDs (24a, 24b) which emit at different wavelengths.

The invention relates to a medical and/or cosmetic radiation device.

A wide variety of devices for exposing human skin to electromagnetic radiation for healing purposes and/or cosmetic purposes are known in the prior art. In this regard, it is known that the effects of electromagnetic radiation (hereinafter referred to as “light” for short, which is also intended to include radiation with wavelengths in the range not visible to the human eye) are greatly dependent on the wavelength of the light.

It is known, for example, that red light produces increased formation of collagen and procollagens in human skin owing to the excitation of fibroblasts. The life of such fibroblasts is increased by the radiation and, overall, an anti-inflammatory effect is achieved. Red light is therefore used particularly also for smoothing wrinkles (so-called skin rejuvenation). Red light is also suitable for wound healing, for so-called photodynamic therapy and for fighting acne.

It is also known that yellow light, similar to red light, acts on fibroblasts and reduces wrinkling owing to increased collagen formation. Furthermore, it is known that under yellow light irradiation porphyrins (metabolic products of acne-causing Propionibacteria) form free oxygen radicals, by which the bacteria are killed. Yellow light is therefore preferably employed in the treatment of acne.

Blue light too has similar and even increased mechanisms of action in relation to acne bacteria, but the depth of penetration into the skin in this case is less than with yellow light.

For medical and/or cosmetic skin treatments with electromagnetic radiation, the light sources primarily used in the prior art are lasers. As is known, lasers have the advantages of monochromatic emission, relatively high power and also the possibility of a locally very accurately targeted localisation of the irradiation. This possibility of accurate adjustment of the wavelength with relatively small bandwidth has proved particularly effective with specific indications and treatments, since the application structures can be specifically selected and the desired effect can be achieved particularly quickly and without detrimental side effects. With the high power and intensity of the laser radiation, it is usually also easy to achieve irradiation intensities on the skin region intended for treatment which exceed the intensity thresholds required for the desired healing effect. These intensity thresholds are normally referred to in the prior art as “fluence” and refer to a radiation power density required for the effectiveness of the treatment.

Nevertheless, laser systems are generally disadvantageous in that they are of relatively complex construction and thus generally require specifically trained staff for operation and maintenance. Lasers are also relatively expensive and frequently also constitute safety hazards. Their use in medical practices is therefore possible only to a limited extent and, as a rule, requires special safety measures.

Where radiation of different wavelength is to be employed at the same application site using laser systems, the outlay on apparatus increases considerably.

Recently, simpler and thus less expensive IPL flashlamp systems (IPL=Incoherent Pulsed Lamp) have become widespread on the market as an alternative to the relatively expensive laser systems. Such multichromatic light sources likewise achieve quite high fluence values with a correspondingly high electrical power supply, and these values enable a series of medical and cosmetic applications. However, the radiation of IPL systems is not very well directed, so that without special additional equipment these systems allow rather only large-area treatment which is locally relatively unspecific. IPL systems also generally emit very wideband radiation, so that when irradiation is to be performed with selected wavelengths, expensive filter arrangements are required. Moreover, the monochromatism of the radiation is generally only inadequately successful. A further disadvantage of IPL systems is that a large part of the emission is heat radiation. This not only has disadvantages in terms of the patient's exposure to IR radiation, but also requires a high degree of cooling of the lamp systems themselves. A further disadvantage of the known IPL systems is that they require high voltage, thereby constituting a safety hazard which necessitates expensive safety measures. Furthermore, the known IPL systems are bulky and have a relatively high current consumption. Although IPL systems enable the setting of different spectral ranges for skin treatment by a suitable choice of filters, the setting of different wavelengths with these systems is possible exclusively successively, i.e. it is not possible to use different wavelengths simultaneously for the treatment, without excessive apparatus outlay on optics and filters.

For some years, solid-state light sources with increasingly improved performance data have been available, these normally being referred to as LED light sources (LED=Light Emitting Diode). In particular, so-called HB LEDs (HB=High Brightness) have been available for several years. With such LEDs, the above-mentioned fluence values are achievable for a large number of medical and/or cosmetic applications.

US 2004/0127961 describes a light source for therapeutic or diagnostic purposes having a plurality of LEDs of two different types, which respectively emit radiation in the wavelength range of 370-450 nm and 620-700 nm.

EP 0 320 080 describes a device for biostimulation comprising an array of LEDs which emit in at least three different wavelength ranges. These LEDs may be positioned, in different embodiments, in a way which can be referred to as alternately according to wavelength.

WO 03/001984 describes a method for the photomodulation of living tissue, in which the tissue is exposed to a multichromatic source of electromagnetic radiation having is a small bandwidth under conditions which are effective for the stimulation of the tissue. The teaching of optically manipulating radiation beams such that, overall, the treatment field is homogeneously illuminated is not set out therein.

The object on which the invention is based is to provide a device of the type mentioned at the outset which enables improved and in particular reliable treatment results.

Devices according to the invention for achieving this aim are described in the claims.

According to a preferred configuration of the invention, in a relatively large panel a plurality of LEDs of one wavelength are arranged alternately with a plurality of LEDs of another wavelength. In this case, LEDs of a third wavelength and LEDs of further wavelengths may also be provided.

According to another preferred configuration of the invention, an electronic control, with which the time sequence of the radiation of individual LEDs is selectively controllable, is provided. In particular, one or more of the following parameters may be selectively adjusted with this control: the instants of the radiation pulses, the lengths of the radiation pulses, the intensities of the radiation pulses, the intervals of the radiation pulses, the ratio of the intensities of radiation pulses of different wavelength, and the variation with time of the intensity of the radiation within the individual pulses.

A further advantage of the invention is that LEDs may be arranged in an application head of a hand-held appliance. In this variant of the invention, the doctor or the specialist staff can thus move the hand-held appliance into the vicinity of the patient's skin without the patient having to assume a particular position.

A further variant of the invention provides that at least some of the LEDs are positioned in such a way, and/or in front of at least some of the LEDs optical elements are arranged in such a way that the radiation beams of the LEDs substantially overlap in the application region, in particular homogeneously.

A particular configuration of the invention provides that a plurality of LEDs which emit radiation in wavelengths selectable as desired are arranged on a chip. The radiation beams of these LEDs can then be superposed in a simple and compact manner so as to produce at the desired site of use a homogeneous radiation distribution, i.e. a radiation distribution in which the radiation density over the entire desired irradiation area is equal, i.e. the radiation energy per unit area is equal. In this case, one optical system may be used for all the LEDs.

Exemplary embodiments of the invention are explained in more detail with reference to the drawing, in which:

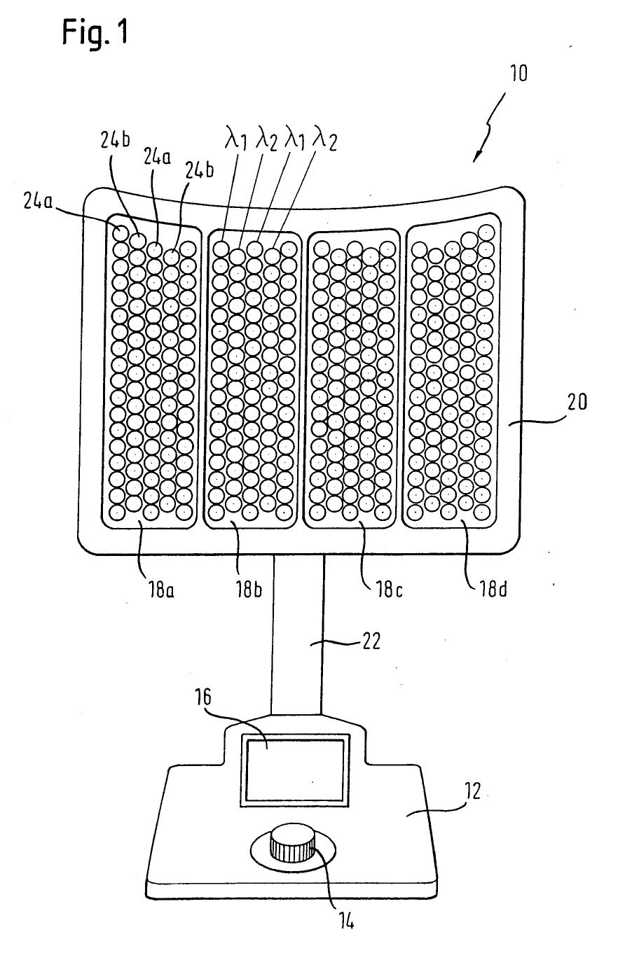

FIG. 1 shows a first exemplary embodiment of a radiation device for medical and/or cosmetic purposes having a multiplicity of LEDs which emit at two different wavelengths;

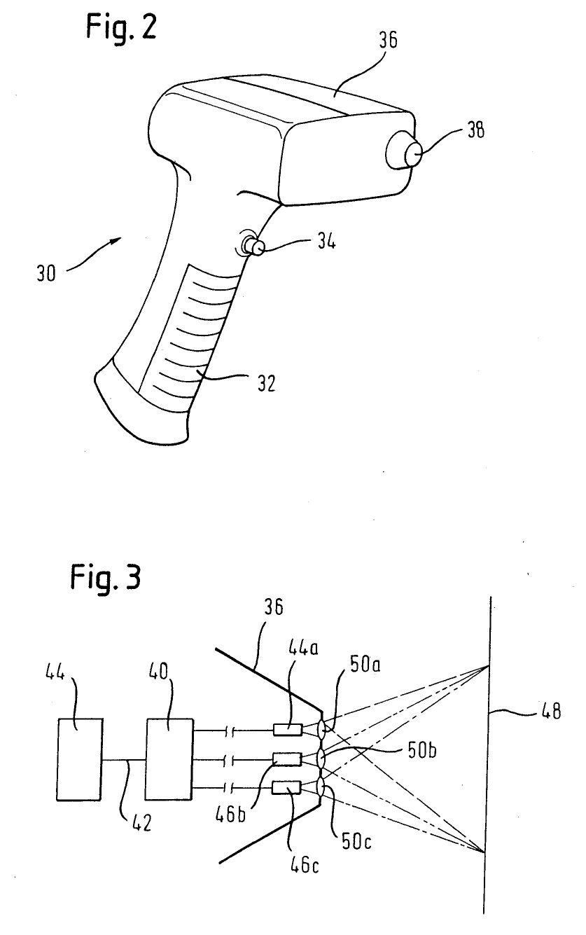

FIG. 2 shows a configuration of the medical and/or cosmetic radiation device as a hand-held appliance;

FIG. 3 shows an arrangement of LEDs, for example in an appliance according to FIG. 2, such that the respectively emitted radiation beams overlap homogeneously;

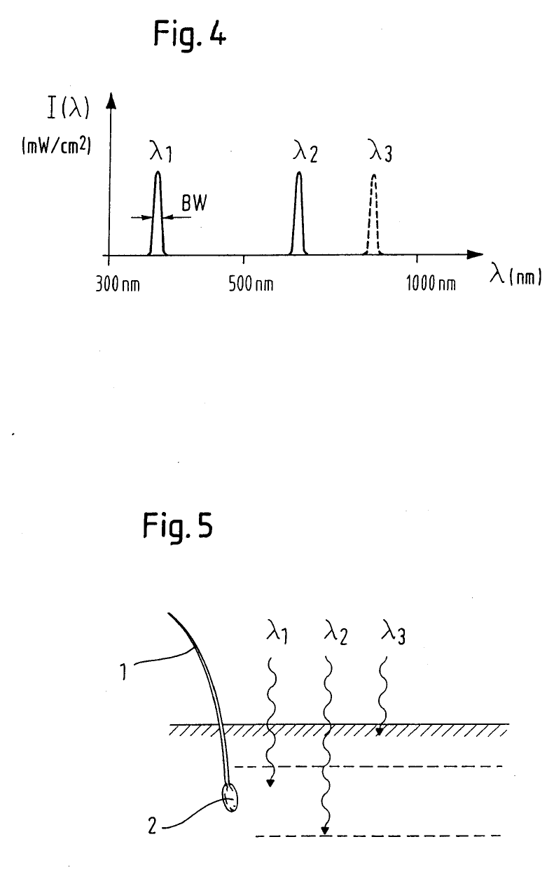

FIG. 4 shows an exemplary embodiment of a time control of the radiation emitted by different LEDs;

FIG. 5 shows, schematically, the depths to which radiation of different wavelength penetrates into human skin; and

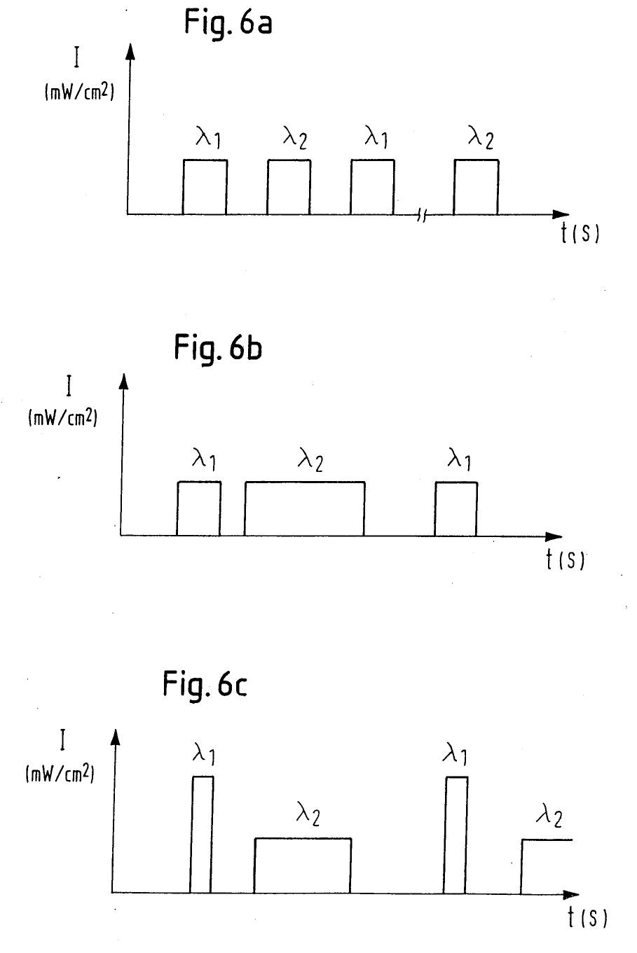

FIG. 6 shows further exemplary embodiments of different time controls of individual LEDs with different emission wavelengths.

The medical or cosmetic radiation device 10 according to FIG. 1 has a base 12 with a control means 14 and a display 16. A plurality of LED arrays 18a, 18b, 18c and 18d are arranged in a panel 20. Each of the said LED arrays has a multiplicity of LEDs. In this exemplary embodiment, the device contains two different LEDs, namely LEDs 24a and LEDs 24b. The LEDs 24a emit light of a first wavelength λ₁, while the LEDs 24b emit light of another wavelength λ₂. As illustrated, the LEDs 24a and 24b are arranged alternately in rows, so that the skin to be irradiated of a patient located in front of the device receives radiation with two different wavelengths, which practically have a single local origin, namely the entire area formed by the LED arrays 18a, 18b, 18c and 18d.

FIG. 2 shows a further exemplary embodiment of a medical or cosmetic radiation device which is designed as a hand-held appliance 30 with a handle 32. An LED array 38, which may consist of a plurality of LEDs with different emitted wavelengths, is arranged on a head 36 of the hand-held appliance 30.

FIG. 3 shows an exemplary embodiment of the way in which an LED array 38 may be configured in a device according to FIG. 2 and FIG. 1. In FIG. 3, only three LEDs 46a, 46b and 46c are illustrated, representatively, but a multiplicity of further LEDs may be realised analogously. The individual LEDs 46a, 46b, 46c are individually selectively activated via an electronic control 40. The electronic control 40 is connected to an operating unit 44 via a line 42. Exemplary embodiments of the time control of the individual LEDs are described in more detail below.

In the arrangement according to FIG. 3, the individual LEDs 46a, 46b, 46c are assigned optical elements 50a, 50b and 50c, respectively, which superpose the radiation beams of the individual LEDs according to FIG. 3 such that a uniform, i.e. homogeneous, illumination takes place in the application plane 48. In addition to the action of the optical elements 50a, b, c, the LEDs 46a, b, c may also be arranged to be inclined relative to one another such that their radiation beams overlap optimally uniformly, with the result that they appear to emanate from the same location in the application plane 48.

FIG. 4 shows an exemplary embodiment of possible different wavelengths which are emitted by the different LEDs. LEDs are capable of producing spectrally quasimonochromatic emissions, for example with the wavelengths λ₁, λ₂ and optionally λ₃ according to FIG. 4. The bandwidth here is typically in the range from 10 to 20 nm. As illustrated, wavelengths from the UV range up to the IR range may be employed. The colour combinations yellow/blue for the treatment of acne and also yellow/red for the combined treatment of acne with rejuvenation, as mentioned at the beginning, are particularly suitable.

FIG. 5 shows, schematically, different penetration depths of radiation with three different wavelengths λ₁, λ₂ and λ₃. In addition, a typical depth of a hair 1 with a hair follicle 2 is shown. This representation illustrates the particular advantage of a radiation device according to the illustrated exemplary embodiments with different wavelengths, with which, depending on the target structures, selectively different penetration depths can be achieved with one and the same device, depending on the control of the individual LEDs.

FIGS. 6a, 6b and 6c shows different time controls of individual LEDs, as may be carried out for example with an electronic control 40 according to FIG. 3. In FIGS. 6a, b, c, the radiation intensities in mW/cm² are plotted on the abscissa and the time, for example in seconds, is plotted on the ordinate.

In a simple configuration, according to the invention the LEDs may be activated simultaneously, i.e. the emitted radiation of different wavelengths simultaneously reaches the skin (not illustrated in FIG. 6). Depending on the medical or cosmetic application, however, the invention enables other simple time controls of different wavelengths, without changing the sources of radiation. All that is necessary is to adjust the time control of the individual LEDs by means of the control 40, i.e. the control 40 contains different control programs which may be selectively chosen by the user with the operating unit 44 and put into operation.

For example, according to FIG. 6a, the individual radiation pulses of different wavelengths λ₁ and λ₂ may be emitted by different diodes alternatingly in respect of time, but with the same intensity. In this case, not only the individual pulse lengths, the pulse-duty factor, and the pulse intensities but also the intervals between the pulses may be adjusted using the control 40. A variation of the aforementioned parameters in the course of a treatment may also be predetermined in advance as being adjustable.

FIG. 6b shows an exemplary embodiment in which the LEDs of one wavelength λ₁ emit radiation with a shorter pulse length than the other LEDs which emit at the wavelength λ₂. Analogously, the intensity can also be selectively adjusted, depending on the application.

FIG. 6c shows an exemplary embodiment in which the control includes a program according to which both the intensity and the length of the individual pulses may be varied as illustrated. It is thus possible, depending on the application, to combine the effect achieved with the wavelength λ₁, which is optimal at a shorter pulse length and higher intensity, with another radiation of wavelength λ₂, which displays an optimal effect at a longer pulse length but lower intensity.

The exemplary embodiments illustrated in FIGS. 6a, b and c can be applied analogously to more than two wavelengths.

The medical or cosmetic radiation devices illustrated are particularly suitable for the following applications: skin rejuvenation, treatment of acne, wound healing, atopic eczema, psoriasis and vitiligo. The appliances may also be employed in phototherapeutic methods in combination with pharmaceuticals.

Finally, the radiation devices described are not only limited to therapeutic purposes, but also enable new diagnostic systems which, owing to the possibility of radiating a plurality of wavelengths in a time-controlled manner, open up particular spectroscopic detection possibilities, in particular the detection of specific absorption bands and also the detection of specific fluorescence.

1.-5. (canceled)

6. Medical or cosmetic radiation device having a hand-held appliance, comprising:
a handle;
a head coupled to said handle and having an LED array which is positionable close to a patient's skin in an application region, said LED array having a plurality of LED's of different wavelengths, and;
optical elements arranged in front of said LED array in such a way, that the radiation beams of the LED's overlap in the application region and a substantially homogeneous illumination takes place in the application region.

7. Device according to claim 6, wherein a plurality of LED's of one wavelength (λ₁) are arranged alternately with LED's of another wavelength (λ₂) in the device.

8. Device according to claim 6, wherein an electronic control, with which the time sequence of the radiation of individual LED is controllable.

9. Device according to claim 8, wherein one or more of the following parameters are selectively adjustable with the electronic control: the lengths of the radiation pluses, the intensities of the radiation pulses, the intervals of the radiation pulses, the ratio of the intensities of radiation pulses of different wavelength, and the variation with time of the intensity of the radiation with the individual pulses.

10. A method of treating the skin of a human patient, comprising:
providing a handheld appliance with an LED array having a plurality of LED's of different individual wavelengths;
positioning the LED array close to the patient's skin; and
directing radiation pulses from the plurality of LED's of different individual wavelengths onto an application region on the patient's skin, wherein the LED energy beams overlap to establish a substantially homogenous illumination on the application region.

11. The method of claim 10, further including controlling the time sequence of radiation pulses for individual LED's within the LED array.

12. The method of claim 10, further including controlling the lengths of the radiation pulses.

13. The method of claim 10, further including controlling the intensities of the radiation pulses.

14. The method of claim 10, further including controlling the intervals of the radiation pulses.

15. The method of claim 10, further including controlling the ratio of intensities of radiation pulses of different wavelengths and the variation with time of the intensity of the radiation with the individual pulses.
